(19) United States
(12) Patent Application Publication
(10) Pub. No.: US 2006/0177403 A1
(43) Pub. Date: Aug. 10, 2006

(54) Cosmetic/dermatological compositions comprising a derivative of glucose and of vitamin F and A specific surfactant

(75) Inventors: Patricia Mezure, Bougival (FR); Claude Dubief, Le Chesnay (FR)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(73) Assignee: L'OREAL, PARIS (FR)

(21) Appl. No.: 11/349,249

(22) Filed: Feb. 8, 2006

Related U.S. Application Data
(60) Provisional application No. 60/672,070, filed on Apr. 18, 2005.

(30) Foreign Application Priority Data
Feb. 8, 2005 (FR) 05/50363

(51) Int. Cl. A61K 8/73

(52) U.S. Cl. 424/70.13

(57) ABSTRACT

Topically applicable cosmetic/dermatological compositions suited for improving the condition of human keratin fibers, notably human hair, especially for reducing or stopping loss and/or inducing and/or stimulating growth and/or density thereof, contain a thus effective amount of (a) at least one derivative of glucose and of vitamin F, solubilized in a topically applicable, physiologically acceptable medium therefor which includes (b) at least one nonionic surfactant having a hydrophilic/lipophilic balance (HLB) of greater than 10, (c) at least one C₁-C₄ alcohol and (d) water.

### CROSS-REFERENCE TO PRIORITY/PROVISIONAL APPLICATIONS

This application claims priority under 35 U.S.C. §119 of FR 05/50363, filed Feb. 8, 2005, and of provisional application Ser. No. 60/672,070, filed Apr. 18, 2005, each hereby expressly incorporated by reference and each assigned to the assignee hereof.

### BACKGROUND OF THE INVENTION

1. Technical Field of the Invention

The present invention relates to cosmetic or dermatological compositions containing, inter alia, a derivative of glucose and of vitamin F and a specific surfactant, for improving the condition of human keratin fibers and especially for reducing or stopping their loss and/or for inducing and/or stimulating their growth.

The present invention also relates to a cosmetic treatment regime or regimen for stimulating the growth of these human keratin fibers and/or for stopping their loss and also to the administration of the combination of the derivative of glucose and of vitamin F and of a specific surfactant for inducing and/or stimulating the growth of these keratin fibers and/or for stopping their loss.

The human keratin fibers to which the invention applies are especially head hair, the eyebrows, the eyelashes, beard hair, moustache hair and pubic hair. The invention applies more especially to human head hair and/or eyelashes.

Thus, more specifically, this invention relates to physiologically acceptable compositions that contain derivatives of glucose and of vitamin F and a particular nonionic surfactant, for improving the condition of human hair and especially for inducing and/or stimulating hair growth and/or for stopping its loss.

2. Description of Background and/or Related and/or Prior Art

In human beings, hair growth and its renewal are mainly determined by the activity of the hair follicles and their dermo-epidermal environment. Their activity is cyclic and essentially comprises three phases, namely, the anagenic phase, the catagenic phase and telogenic phase.

The anagenic phase (active phase or growth phase), which lasts several years and in the course of which the hair gets longer, is followed by a very short and transient catagenic phase which lasts a few weeks. During this phase, the hair undergoes a change, the follicle becomes atrophied and its implantation in the dermis appears higher and higher.

The terminal phase, which lasts a few months, corresponds to a rest phase, known as the telogenic phase. At the end of this rest period, the hairs fall out and a new cycle starts.

The hair is thus in constant renewal and, out of the approximately 150,000 hairs which make up a head of hair, at any moment, about 10% of them are at rest and will thus be replaced within a few months.

The literature provides many reasons leading to early hair loss: in particular, this early hair loss occurs in individuals who are genetically predisposed, and this especially affects men. This more particularly involves androgenetic or androgenic alopecia or alternatively androgeno-genetic alopecia.

This alopecia is essentially due to a disruption in hair renewal, which results, in a first stage, in acceleration of the frequency of the cycles at the expense of the quality and then the quantity of the hairs. A gradual thinning of the head of hair takes place by regression of the so-called “terminal” hairs at the downy stage. Certain regions are preferentially affected, in particular the temporal or frontal areas and the upper part of the occipital area in men, whereas a diffuse alopecia of the vertex is observed in women.

Other causes may lead to a substantial, temporary or permanent loss of hair. It may be a case of loss of or impairment in the hair after pregnancy (post-partum), during states of denutrition or dietary imbalance, or alternatively during states of asthenia or of hormonal dysfunction, as may be the case during or after the menopause. It may also be a case of loss of or impairment in the hair in relation with seasonal phenomena.

Substances for suppressing or reducing the effect of alopecia, and in particular for inducing or stimulating the growth of head hairs and/or certain other hairs, or even for reducing or slowing down hair loss, have been sought for many years, particularly in the cosmetics industry.

In this perspective, a quite specific novel compound, an O-acyl derivative of glucose, exhibits noteworthy properties that justify its use as an active agent for limiting hair loss and/or for promoting its regrowth; this compound corresponds to the result of the esterification of glucose with vitamin F. Its synthesis is described in FR-A-2,840,903, in which are illustrated compositions especially containing the derivative of glucose and of vitamin F in an aqueous-alcoholic medium.

The formulation of this derivative in a cosmetic or dermatological composition should satisfy a certain number of technical constraints in order for it to be able to act efficiently as a hair-loss counteractant/hair regrowth active agent. Thus, this derivative should be fully dissolved in the composition. This composition, which is generally used without rinsing, should be easy to apply to all of the part of the scalp to be treated, should cause no irritation and should not be tacky. Furthermore, the hair that may be impregnated with the composition should have a pleasant feel (neither coarse, nor greasy, nor tacky, etc.) and a clean and volumized appearance.

### SUMMARY OF THE INVENTION

After extensive studies, it has now been discovered that these technical constraints are solved or ameliorated by formulating this derivative in a composition containing a nonionic surfactant having a hydrophilic-lipophilic balance (HLB) of greater than 10 and a C₁-C₄ alcohol.

The present invention thus features physiologically acceptable compositions comprising

(a) at least one derivative of glucose and of vitamin F,

(b) at least one nonionic surfactant with a hydrophilic/lipophilic balance of greater than 10,

(c) at least one C₁-C₄ alcohol, and

(d) water.

These compositions are, in particular, suited for inducing and/or stimulating the growth of keratin fibers and/or for stopping their loss and/or for increasing their density. They may be used in unmodified form or added to a more complex physiologically acceptable composition.

The present invention also features the administration of the combination of at least one derivative of glucose and of vitamin F, at least one nonionic surfactant with a hydrophilic/lipophilic balance of greater than 10, at least one C₁-C₄ alcohol and water, in a composition for inducing and/or stimulating the growth of human keratin fibers and/or for stopping their loss and/or for increasing their density. The term “increasing the density of keratin fibers,” and especially hair density, means increasing the number of keratin fibers, and especially of head hairs, per cm² of skin such as the scalp. Thus, such a composition makes it possible to keep the head of hair in good condition and/or to combat the natural loss of head hair, in particular of men.

In particular, this invention relates to the cosmetic formulation of the combination of at least one derivative of glucose and of vitamin F, at least one nonionic surfactant with a hydrophilic/lipophilic balance of greater than 10, at least one C₁-C₄ alcohol and water, into compositions, especially cosmetic compositions for human hair care, for treating alopecia of natural origin and in particular androgenic or androchronogenetic alopecia, and also to the formulation of this combination for the preparation of compositions for caring for and/or treating human keratin fibers, and especially the hair, which are suited to induce and/or stimulate the growth of keratin fibers, and especially the hair, and/or to stop their loss and/or to increase their density.

The present invention also features the cosmetic administration of a human haircare composition, comprising at least one derivative of glucose and of vitamin F, at least one nonionic surfactant with an HLB of greater than 10, at least one C₁-C₄ alcohol and water, for reducing hair loss and/or for increasing its density and/or for treating androgenic alopecia.

This invention also features a cosmetic regime or regimen for treating human keratin fibers and/or the skin from which the said fibers emerge, in particular the scalp, which is especially suited for stimulating the growth of the said human keratin fibers, in particular human head hair and certain other hairs, and/or for stopping their loss, comprising topically applying to these human keratin fibers and/or the skin from which these fibers emerge a cosmetic composition as defined above, maintaining the composition in contact with these keratin fibers and/or the skin from which these fibers emerge, and optionally rinsing these keratin fibers and/or the said skin.

This treatment process does indeed have the characteristics of a cosmetic process insofar as it makes it possible to improve the aesthetic appearance of keratin fibers and in particular of human head hair and eyelashes by giving them greater vigor and an improved appearance. In addition, it may be used daily for several months, without medical prescription.

More especially, the present invention features a cosmetic regime or regimen for caring for human head hair and/or the scalp, in order to improve their condition and/or appearance, comprising topically applying to human head hair and/or the scalp a cosmetic composition as defined above, maintaining the composition in contact with the head hair and/or the scalp, and optionally rinsing the head hair and/or the scalp.

### DETAILED DESCRIPTION OF BEST MODE AND SPECIFIC/PREFERRED EMBODIMENTS OF THE INVENTION

According to the invention, the term “at least one” means one or more (2, 3 or more). In particular, the composition may contain one or more derivatives of glucose and of vitamin F.

In the text that follows, unless otherwise indicated, the amounts of the various ingredients of the composition are given as weight percentages relative to the total weight of the composition.

Starting Materials Essential to the Invention:

Derivative of Glucose and of Vitamin F:

The derivative of glucose and of vitamin F is an O-acyl derivative obtained by partial or total esterification of vitamin F with glucose, the manufacture of which is described in EP-A-1,371,658. This derivative may be represented by a compound of formula (I) below or a mixture of compounds of formula (I): 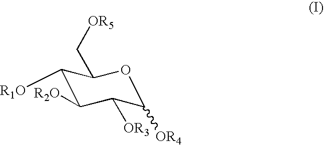
in which R₁, R₂, R₃, R₄ and R₅, which may be identical or different, are each hydrogen or a radical —CO—R wherein R is a saturated or unsaturated, linear hydrocarbon-based radical having 11 to 21 carbon atoms, with the proviso that at least one of the radicals R₁ to R₅ is other than hydrogen.

In particular, the derivative of glucose and of vitamin F comprises a mixture of C₁₆-C₁₈ fatty acid monoesters or diesters. The fatty acid is selected in particular from among linoleic acid, oleic acid, palmitic acid and stearic acid, and mixtures thereof. This derivative of glucose and of vitamin F is especially selected from among the compounds 6-O-octadeca-9-enoyl-D-glucopyranose, 6-O-hexadecanoyl-D-glucopyranose, 6-O-octadeca-9,12-dienoyl-D-glucopyranose, 6-O-octadecanoyl-D-glucopyranose and 3-O-octadeca-9,12-dienoyl-D-glucopyranose, and mixtures thereof.

The derivative is especially the ester of glucose and of vitamin F, predominantly esterified at position 6, obtained in accordance with Example 1 of EP-A-1,371,658.

The amount used of derivative of glucose and of vitamin F corresponds to the amount required to obtain the desired result (i.e., in particular increasing the density of keratin fibers or promoting their growth). One skilled in this art is thus capable of evaluating this effective amount, which depends on the derivative(s) used, the individual on whom it is applied, and the time of this application.

To provide an order of magnitude, in the compositions according to the invention, the derivative of vitamin F and of glucose or the mixture of derivatives of vitamin F and of glucose may be administered in an amount representing from 0.01% to 10% by weight, preferentially 0.05% to 5% by weight and more preferentially from 0.1% to 2% by weight.

Nonionic Surfactants:

The compositions according to the invention contain one or more nonionic surfactants having a hydrophilic-lipophilic balance (HLB) of greater than 10 (see Griffin, W. C., J. Soc. Cosmet. Chem., 1949, 1, 311, and 1954, 5, 249) and which may be up to 20; these are compounds that are well known per se (see especially in this regard “Handbook of Surfactants” by M. R. Porter, published by Blackie & Son (Glasgow and London), 1991, pp. 116-178). Thus, they may be selected especially from among polyethoxylated, polypropoxylated or polyglycerolated fatty acids, (C₁-C₂₀)alkylphenols, alpha-diols and alcohols, which are preferably hydrogenated, with a fatty chain containing, for example, from 8 to 22 (especially from 12 to 18) carbon atoms, the mean number of ethylene oxide or propylene oxide structural units optionally ranging especially from 3.5 to 200 (for example from 5 to 100) and the number of glycerol groups optionally ranging especially from 2 to 100 (for example from 3 to 50), and mixtures thereof.

Also exemplary are copolymers of ethylene oxide and propylene oxide, condensates of ethylene oxide and propylene oxide on fatty alcohols; polyethoxylated fatty amides and preferably those containing on average from 3.5 to 200 mol (for example from 5 to 100 mol) of propylene oxide and/or ethylene oxide; polyglycerolated fatty amides and preferably those containing on average from 1.5 to 40 and in particular from 1.5 to 25 glycerol groups; ethoxylated fatty acid esters of sorbitan especially containing from 2 to 30 mol on average of ethylene oxide and a fatty chain especially containing from 8 to 22 (for example from 12 to 18) carbon atoms; fatty acid esters of sucrose; fatty acid esters of polyethylene glycol; (C₆-C₂₄)alkylpolyglycosides; N—(C₆-C₂₄)alkylglucamine derivatives; amine oxides such as (C₁₀-C₁₄)alkylamine oxides; and mixtures thereof. The term “fatty acids” means monoacids or polyacids containing from 8 to 22 and preferably from 12 to 18 carbon atoms.

According to one embodiment, one or more surfactants selected from among hydrogenated, polyethoxylated, polypropoxylated or polyglycerolated fatty acids, (C₁-C₂₀)alkylphenols, alpha-diols and alcohols, with a fatty chain containing from 8 to 18 carbon atoms, the mean number of ethylene oxide or of propylene oxide groups ranging from 3.5 to 200 and the number of glycerol groups ranging from 2 to 100, and mixtures thereof, are used as nonionic surfactant with an HLB of greater than 10.

The nonionic surfactant or the mixture of nonionic surfactants with an HLB of greater than 10 is (are) preferably used in an amount sufficient to dissolve with the alcohol the derivative of vitamin F and of glucose. Its content thus depends on the content of derivative of vitamin F and of glucose. In practice, this nonionic surfactant or mixture of nonionic surfactants may be included in the compositions of the invention in a concentration ranging from 0.01% to 10% by weight, preferentially from 0.05% to 5% by weight and more preferentially from 0.1% to 2% by weight.

The Alcohol:

It may be used alone or as a mixture and is selected from among C₁-C₄ alcohols such as ethanol or isopropanol, and mixtures thereof. The C₁-C₄ alcohol or the mixture of C₁-C₄ alcohols is preferably included in an amount sufficient to dissolve with the nonionic surfactant the derivative of vitamin F and of glucose. Its content thus depends on the content of derivative of vitamin F and of glucose. In practice, the C₁-C₄ alcohol or the mixture of C₁-C₄ alcohols may be included in the compositions of the invention in a concentration ranging from 2% to 80% of the total weight of the composition, preferentially from 10% to 70% and more preferentially from 20% to 60% by weight relative to the total weight of the composition.

The compositions of the invention may be for cosmetic or pharmaceutical applications. The compositions of the invention are preferentially for cosmetic applications and in particular for topical application to the skin and keratin fibers, and more especially to the human scalp, head hair and eyelashes. Thus, the composition should contain a non-toxic physiologically acceptable medium that can be applied to human skin, including the scalp and the eyelids, and keratin fibers.

The compositions may be in any known galenical form suitable for the mode of administration.

Starting Materials Not Essential to the Invention:

Galenical Form:

For a topical application to the skin, including the scalp, the composition may be in the form of a solution, a suspension, an emulsion or a dispersion of more or less fluid consistency and especially liquid or semi-liquid consistency, obtained by dispersing a fatty phase in an aqueous phase (O/W) or, conversely, (W/O), or alternatively a gel.

A composition in the form of a mousse or in the form of a spray or an aerosol then comprising a pressurized propellant may also be provided.

In particular, the compositions for application to the scalp or the hair may be in the form of a haircare lotion, for example for daily or twice-weekly application, a shampoo or hair conditioner, in particular for twice-weekly or weekly application, a liquid or solid soap for cleansing the scalp, for daily application, a hairstyle shaping product (lacquer, hairsetting product or styling gel), a treating mask, or a foaming cream or gel for cleansing the hair. They may also be in the form of a hair dye or hair mascara to be applied with a brush or a comb.

Moreover, for application to the eyelashes or other hairs, the compositions of the invention may be in the form of a pigmented or non-pigmented mascara, to be applied with a brush to the eyelashes or alternatively to beard or moustache hair.

According to one particular embodiment, the compositions according to the invention are in the form of a hair cream or hair lotion, a shampoo or hair conditioner, or a mascara for the hair or the eyelashes.

The amounts of the various constituents of the physiological medium of the composition according to the invention are those generally included in the fields under consideration. In addition, these compositions are prepared according to the usual methods.

When the composition is an emulsion, the proportion of the fatty phase may range from 2% to 80% by weight and preferably from 5% to 50% by weight relative to the total weight of the composition. The aqueous phase is adjusted as a function of the content, in the fatty phase, of derivative of vitamin F and of glucose, of alcohol and nonionic surfactant and also that of the optional additional ingredients, to obtain 100% by weight. In practice, the aqueous phase represents from 5% to 89.9% by weight.

The fatty phase may contain fatty or oily compounds, which are liquid at room temperature (25° C.) and atmospheric pressure (760 mmHg), generally known as oils, waxes and pasty or semi-solid products. These oils may be mutually compatible and may form a macroscopically homogeneous liquid fatty phase.

The aqueous phase contains water and optionally a water-miscible ingredient, for instance polyols such as propylene glycol, glycerol or sorbitol.

Formulation Additives:

The compositions of the invention may also comprise one or more other ingredients usually employed in the fields under consideration, selected from among formulation additives, for instance aqueous-phase or oily-phase thickeners or gelling agents, dyestuffs that are soluble in the medium of the composition, solid particles such as mineral or organic fillers or pigments in the form of microparticles or nanoparticles, preservatives, fragrances, hydrotopes or electrolytes, neutralizers (acidifying or basifying agents), propellants, anionic, cationic or amphoteric surfactants, polymers, in particular water-soluble or water-dispersible anionic, nonionic, cationic or amphoteric film-forming polymers, mineral or organic salts, chelating agents; mixtures thereof. These additives may be present in the composition in the amounts generally employed in cosmetics and dermatology, and especially in a proportion of from 0.01% to 50% and preferably from 0.1% to 20%, for example from 0.1% to 10%, of the total weight of the composition.

Needless to say, one skilled in this art will take care to select the optional additional ingredients and/or the amount thereof such that the advantageous properties of the compositions according to the invention, i.e., especially the increase in the density of the keratin fibers, are not, or are not substantially, adversely affected by the envisaged addition.

As oils that may be included according to the invention, mention may be made of oils of mineral origin (liquid petroleum jelly or hydrogenated isoparaffin), oils of plant origin (liquid fraction of shea butter, sunflower oil, apricot oil, soybean oil, fatty alcohol or fatty acid), oils of animal origin (perhydrosqualene), synthetic oils (fatty acid esters or purcellin oil), silicone oils (linear or cyclic polydimethylsiloxanes, and phenyl trimethicones) and fluoro oils (perfluoropolyethers). Waxes that may be mentioned include silicone waxes, beeswax, candelilla wax, rice bran wax, carnauba wax, paraffin wax or polyethylene wax.

When the composition is in emulsion form, it also contains one or more emulsifiers and optionally one or more co-emulsifiers generally employed in cosmetics and pharmaceuticals. Their nature also depends on the sense of the emulsion. In practice, the emulsifier and, optionally, the co-emulsifier are present in the composition in a proportion ranging from 0.1% to 30% by weight, preferably from 0.5% to 20% by weight and better still from 1% to 8% by weight. The emulsion may also contain lipid vesicles and especially liposomes.

As thickeners or gelling agents that may be included according to the invention, mention may be made of thickening or gelling polymers, for instance crosslinked polyacrylic acids, associative polymers and non-polymeric thickeners or gelling agents, for instance modified or unmodified clays, amides and metal salts of fatty acids.

Cosmetic and Pharmaceutical Active Agents:

The compositions of the invention may also comprise one or more cosmetic and pharmaceutical active agents with beneficial action on the skin, and especially on the scalp and keratin fibers (other than the derivative of vitamin F and of glucose). As cosmetic or pharmaceutical active agents with beneficial action on the hair or the scalp, which may be included according to the invention, mention may be made of active agents selected from among:

UV-blocking agents, such as sunscreens;

vitamins (A, C or E) and derivatives thereof (retinyl palmitate, tocopheryl acetate or tocopheryl palmitate);

ceramides;

proteins and protein hydrolysates, peptides and amino acids (natural);

urea and allantoin;

sugars and sugar derivatives, for instance reduced or oxidized sugars;

extracts of plant origin (those from Iridacea plants or from soybean) or of bacterial origin;

hydroxy acids, in particular hydroxycarboxylic acids or ketocarboxylic acids (fruit acid, salicylic acid) and esters thereof, for instance 5-n-octanoylsalicylic acid;

diazoxide, spiroxazone, or phospholipids, for instance lecithin, especially defatted lecithin;

additional active compounds that promote regrowth of the hair and/or limit hair loss, such as, especially: nicotinic acid esters, among which especially are tocopheryl nicotinate, benzyl nicotinate and C₁-C₂₂ alkyl nicotinates such as methyl or hexyl nicotinate; pyrimidine derivatives, for instance 2,4-diamino-6-piperidinopyrimidine 3-oxide or “Minoxidil” described in U.S. Pat. Nos. 4,139,619 and 4,592,812; pyrimidine 3-oxide derivatives, for instance those described in WO 92/01437 or WO 96/09048, and especially “Aminexil” or 2,4-diaminopyrimidine 3-N-oxide; anti-androgens, for instance steroidal or non-steroidal inhibitors of 5-α-reductase, such as finasteride and the compounds described in U.S. Pat. No. 5,516,779, cyprosterone acetate, azeleic acid, its salts and its derivatives and the compounds described in U.S. Pat. No. 5,480,913, flutamide and the compounds described in U.S. Pat. Nos. 5,411,981, 5,565,467 and 4,910,226, prostaglandins, for instance PGF-2α or PGE2 in the form of salts or esters, and analogues thereof, for instance latanoprost;

anti-bacterial, anti-fungal or anti-dandruff agents, for instance selenium derivatives, ketoconazole, octopirox, triclocarban, triclosan, zinc pyrithione, itraconazole, asiatic acid, hinokitiol, mipirocine, tetracyclines, especially erythromycin and the compounds described in EP-0,680,745, clinycine hydrochloride, benzoyl peroxide or benzyl peroxide and minocycline;

calcium-channel antagonists and potassium-channel agonists;

hormones;

steroidal anti-inflammatory agents, for instance glucocorticoids, corticosteroids (for example: hydrocortisone) and non-steroidal anti-inflammatory agents, for instance glycyrrhetinic acid and α-bisabolol, benzydamine and the compounds described in EP-0,770,399, WO 94/06434 and FR-2,268,523;

retinoid RXR receptor agonists and retinoid antagonists;

free-radical scavengers and antioxidants, for instance butylhydroxyanisole or butylhydroxytoluene;

anti-seborrhoeic agents;

anti-parasitic agents;

anti-viral agents;

anti-pruriginous agents;

carotenoids, for instance β-carotene;

lactones and the corresponding salts thereof;

essential fatty acids, for instance linoleic acid, eicosatetraenoic acid, linolenic acid and eicosatrienoic acid, or esters and amides thereof;

essential oils;

phenols and polyphenols, for instance flavonoids;

mixtures thereof.

According to one preferred embodiment of the invention, the composition also contains an additional active agent that promotes regrowth of the hair and/or that limits hair loss, and in particular minoxidil, aminexil or latanoprost, or mixtures thereof. This additional active agent may be in an amount that is effective for reducing hair loss and/or limiting hair loss and/or for increasing its density, for example in an amount of from 0.001% to 10% by weight, preferably from 0.1% to 5% and better still from 0.5% to 3% by weight.

In order to further illustrate the present invention and the advantages thereof, the following specific examples are given, it being understood that same are intended only as illustrative and in nowise limitative.

### EXAMPLES:

Demonstration:

(a) The four compositions below (in grams) are prepared:

| | Composition | | | |
|---|---|---|---|---|
| | A | B | C | D |
| Derivative of vitamin F and of | 5 | 5 | 5 | 5 |
| glucose* | | | | |
| PEG 40 hydrogenated castor | | 3 | | 3 |
| oil | | | | |
| Ethanol | 24.6 | 24.6 | 42.4 | 42.4 |
| Water | qs 100 | qs 100 | qs 100 | qs 100 |
| Appearance of the | milky | clear | clear | clear |
| composition (T0) | | | | |
| Appearance of the | milky | clear | milky | clear |
| composition (D0 + 1) | | | | |

*obtained according to Example 1 of EP-A-1-371,658

These tests fully illustrate the role of the nonionic surfactant on the solubility of the active agent.

(b) 1 ml of freshly prepared compositions B, C and D are separately applied to three 2.5 g locks of natural hair.

On application, the lock treated with composition C is very coarse and is difficult to disentangle. The locks treated with compositions B and D are soft and easy to disentangle. After drying, the feel and the appearance of the hair treated with compositions B and D are better than with composition C. They feel smooth, disentangle easily without developing static electricity, and are shiny.

These tests illustrate the beneficial effect of the surfactant on the cosmetic properties of the hair.

Example of a Composition According to the Invention:

A serum is prepared, having the composition:

| | | |
|---|---|---|
| Derivative of vitamin F and of glucose* | 0.1 | g |
| Aminexil | 1.5 | g |
| PEG 40 hydrogenated castor oil (HLB 13) | 0.1 | g |
| Ethanol | 47.2 | g |
| Water | qs 100 | g |

The composition is in the form of a totally transparent colorless fluid lotion.

It applies very easily to the scalp and is easy to distribute. It dries rapidly without causing any discomfort. The hair impregnated with the composition is soft and smooth.

Each patent, patent application, publication and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A topically applicable cosmetic/dermatological composition for improving the condition of human keratin fibers, comprising a thus effective amount of (a) at least one derivative of glucose and of vitamin F, solubilized in a topically applicable, physiologically acceptable medium therefor which comprises (b) at least one nonionic surfactant having a hydrophilic/lipophilic balance (HLB) of greater than 10, (c) at least one C₁-C₄ alcohol and (d) water.

2. The cosmetic/dermatological composition as defined by claim 1, said at least one derivative of glucose and of vitamin F being an O-acyl derivative prepared by partial or total esterification of vitamin F with glucose.

3. The cosmetic/dermatological composition as defined by claim 1, said at least one derivative of glucose and of vitamin F having the formula (I): 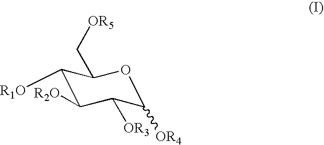
in which R₁, R₂, R₃, R₄ and R₅, which may be identical or different, are each hydrogen or a radical —CO—R wherein R is a saturated or unsaturated, linear hydrocarbon-based radical having 11 to 21 carbon atoms, with the proviso that at least one of the radicals R₁ to R₅ is other than hydrogen.

4. The cosmetic/dermatological composition as defined by claim 1, said at least one derivative of glucose and of vitamin F being selected from the group consisting of 6-O-octadeca-9-enoyl-D-glucopyranose, 6-O-hexadecanoyl-D-glucopyranose, 6-O-octadeca-9,12-dienoyl-D-glucopyranose, 6-O-octadecanoyl-D-glucopyranose, 3-O-octadeca-9,12-dienoyl-D-glucopyranose, and mixtures thereof.

5. The cosmetic/dermatological composition as defined by claim 1, said at least one derivative of glucose and of vitamin F comprising from 0.01% to 10% by weight thereof.

6. The cosmetic/dermatological composition as defined by claim 1, said at least one derivative of glucose and of vitamin F comprising from 0.1% to 2% by weight thereof.

7. The cosmetic/dermatological composition as defined by claim 1, said at least one nonionic surfactant having an HLB of greater than 10 being selected from the group consisting of polyethoxylated, polypropoxylated or polyglycerolated fatty acids, (C₁-C₂₀)alkylphenols, alpha-diols and alcohols, copolymers of ethylene oxide and propylene oxide, condensates of ethylene oxide and propylene oxide on fatty alcohols; polyethoxylated fatty amides; polyglycerolated fatty amides; ethoxylated fatty acid esters of sorbitan; fatty acid esters of sucrose; fatty acid esters of polyethylene glycol; (C₆-C₂₄)alkylpolyglycosides; N-(C₆-C₂₄)alkylglucamine derivatives; amine oxides; and mixtures thereof.

8. The cosmetic/dermatological composition as defined by claim 1, said at least one nonionic surfactant having an HLB of greater than 10 being selected from the group consisting of hydrogenated, polyethoxylated, polypropoxylated or polyglycerolated fatty acids, (C₁-C₂₀)alkylphenols, alpha-diols and alcohols, with a fatty chain having from 8 to 18 carbon atoms, the mean number of ethylene oxide or of propylene oxide groups ranging from 3.5 to 200 and the number of glycerol groups ranging from 2 to 100, and mixtures thereof.

9. The cosmetic/dermatological composition as defined by claim 1, said at least one nonionic surfactant or mixture of nonionic surfactants having an HLB of greater than 10 comprising an amount sufficient to dissolve with the alcohol the derivative of vitamin F and of glucose.

10. The cosmetic/dermatological composition as defined by claim 1, said at least one nonionic surfactant or mixture of nonionic surfactants having an HLB of greater than 10 comprising from 0.01% to 10% by weight thereof.

11. The cosmetic/dermatological composition as defined by claim 1, said at least one nonionic surfactant or mixture of nonionic surfactants having an HLB of greater than 1 0 comprising from 0.1% to 2% by weight thereof.

12. The cosmetic/dermatological composition as defined by claim 1, said at least one C₁-C₄ alcohol being selected from the group consisting of ethanol, isopropanol, and mixtures thereof.

13. The cosmetic/dermatological composition as defined by claim 1, said at least one C₁-C₄ alcohol or mixture of such alcohols comprising from 2% to 80% by weight thereof.

14. The cosmetic/dermatological composition as defined by claim 1, said at least one C₁-C₄ alcohol or mixture of such alcohols comprising from 2% to 60% by weight thereof.

15. The cosmetic/dermatological composition as defined by claim 1, formulated as a hair cream or hair lotion, a shampoo or hair conditioner, or a mascara for the hair or the eyelashes.

16. The cosmetic/dermatological composition as defined by claim 1, further comprising one or more other ingredients selected from the group consisting of aqueous-phase or oily-phase thickeners or gelling agents, dyestuffs that are soluble in the medium of the composition, mineral or organic fillers and pigments in the form of microparticles or nanoparticles, preservatives, fragrances, hydrotopes, electrolytes, neutralizers, propellants, anionic, cationic or amphoteric surfactants, water-soluble or water-dispersible anionic, nonionic, cationic or amphoteric film-forming polymers, mineral or organic salts, chelating agents; and mixtures thereof.

17. The cosmetic/dermatological composition as defined by claim 1, further comprising one or more cosmetic or pharmaceutical active agents eliciting beneficial action on the hair or the scalp, selected from the group consisting of UV-blocking agents; vitamins and derivatives thereof; ceramides; proteins and protein hydrolysates, peptides and amino acids; urea, allantoin; sugars and sugar derivatives; extracts of plant or bacterial origin; hydroxy acids and esters thereof; diazoxide, spiroxazone or phospholipids; nicotinic acid esters; minoxidil; aminexil; steroidal or non-steroidal inhibitors of 5α-reductase; anti-bacterial, anti-fungal or anti-dandruff agents; calcium-channel antagonists; potassium-channel agonists; hormones; steroidal and non-steroidal anti-inflammatory agents; retinoid RXR receptor agonists and retinoid antagonists; free-radical scavengers and antioxidants; anti-seborrhoeic agents; anti-parasitic agents; anti-viral agents; anti-pruriginous agents; carotenoids; lactones and the corresponding salts thereof; essential fatty acids or esters and amides thereof; essential oils; phenols and polyphenols; and mixtures thereof.

18. The cosmetic/dermatological composition as defined by claim 1, further comprising an additional active agent that promotes regrowth of the hair and/or that limits hair loss.

19. The cosmetic/dermatological composition as defined by claim 1, further comprising an additional active agent that promotes regrowth of the hair and/or that limits hair loss, selected from the group consisting of minoxidil, aminexil, latanoprost, and mixtures thereof.

20. A regime or regimen for inducing and/or stimulating the growth of human keratin fibers and/or for reducing or stopping the loss and/or increasing the density thereof, comprising topically applying thereon or on the area of the skin from which said fibers emerge, a thus effective amount of a composition containing (a) at least one derivative of glucose and of vitamin F, solubilized in a topically applicable, physiologically acceptable medium therefor which comprises (b) at least one nonionic surfactant having a hydrophilic/lipophilic balance (HLB) of greater than 10, (c) at least one C₁-C₄ alcohol and (d) water.

21. A regime or regimen for inducing and/or stimulating the growth of human hair and/or for reducing or stopping the loss and/or increasing the density thereof, comprising topically applying thereon or on the area of the skin from which said human hair emerges, a thus effective amount of a composition containing (a) at least one derivative of glucose and of vitamin F, solubilized in a topically applicable, physiologically acceptable medium therefor which comprises (b) at least one nonionic surfactant having a hydrophilic/lipophilic balance (HLB) of greater than 10, (c) at least one C₁-C₄ alcohol and (d) water.

22. A regime or regimen for treating androgenic alopecia, comprising topically applying onto the affected skin area of an individual in need of such treatment, a thus effective amount of a composition containing (a) at least one derivative of glucose and of vitamin F, solubilized in a topically applicable, physiologically acceptable medium therefor which comprises (b) at least one nonionic surfactant having a hydrophilic/lipophilic balance (HLB) of greater than 10, (c) at least one C₁-C₄ alcohol and (d) water.

23. The regime or regimen as defined by claim 20, including maintaining said composition in contact with said human keratin fibers and/or said area of the skin from which said fibers emerge, and optionally rinsing said human keratin fibers and/or said area of the skin from which said fibers emerge.

24. The regime or regimen as defined by claim 21, including maintaining said composition in contact with said human hair and/or said area of the skin from which said human hair emerges, and optionally rinsing said human hair and/or said area of the skin from which said human hair emerges.
